(19) United States
(12) Patent Application Publication
(10) Pub. No.: US 2025/0230374 A1
(43) Pub. Date: Jul. 17, 2025

(54) DUAL-USE COOLANT AND LUBRICANT

(71) Applicant: CUMMINS INC., Columbus, IN (US)

(72) Inventors: Justin W. Perry, Bloomington, IN (US); Jonathan A. Groeper, Bargersville, IN (US); Corey W. Trobaugh, Southlake, TX (US)

(73) Assignee: CUMMINS INC., Columbus, IN (US)

(21) Appl. No.: 18/698,990

(22) PCT Filed: Oct. 24, 2022
(86) PCT No.: PCT/US2022/047586

§ 371 Date: Apr. 5, 2024

Related U.S. Application Data
(60) Provisional application No. 63/271,539, filed on Oct. 25, 2021.

(51) Int. Cl. C10M 105/12; C07C 35/08; C09K 5/10; C10N 40/16

(52) U.S. Cl. CPC C10M 105/12; C07C 35/08; C09K 5/10; C10M 2207/0215; C10N 2040/16

(57) ABSTRACT

A dual-use coolant includes a fluid. The fluid has the chemical formula of a tertiary alcohol with a R₁, a R₂, and a R₃ group having the following formula (I). The coolant may also be used as a base fluid for a lubricant. The use of a single fluid for both lubrication and cooling may decrease the complexity, weight, and manufacturing costs of the associated system.

### CROSS-REFERENCE TO RELATED APPLICATIONS

This application claims priority to and the benefit of U.S. Provisional Patent Application No. 63/271,539, filed Oct. 25, 2021, the content of which is incorporated herein by reference.

### TECHNICAL FIELD

The present disclosure relates generally to fluids for use in electrical systems such as power electronics, data center cooling, fuel cell stacks, etc.

### BACKGROUND

Current powertrain and/or battery systems use one fluid for lubrication and another fluid for cooling. The use of two separate fluids ultimately increases the complexity, weight, and manufacturing costs of the vehicle due to the need for additional plumbing, pumps, heat sinks, and other components. Furthermore, both fluids require a high, stable breakdown voltage, low electrical dissipation factors, and good thermal properties as well as being inert to electronics and minimally reactive with battery electrolyte.

### SUMMARY

One embodiment relates to a dual-use coolant comprising a fluid (e.g., base fluid). The fluid has the following chemical formula corresponding to Structure I below.

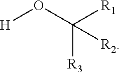

In one embodiment, R₁ in Structure I is a C₍₃₋₂₀₎ alkyl group.

### BRIEF DESCRIPTION OF THE DRAWINGS

The details of one or more implementations are set forth in the accompanying drawings and the description below. Other features, aspects, and advantages of the disclosure will become apparent from the description, the drawings, and the claims, in which:

FIG. 1 is a schematic flow diagram for a method of forming a lubricant, in accordance with some embodiments.

FIG. 2 illustrates chemical structures of oxidation products of various coolants.

FIG. 3 is simulation of inverter temperature on a transit bus from an ethylene-glycol coolant and two dual-use coolants.

FIG. 4 is a table of the properties of various coolants.

It will be recognized that the figures are schematic representations for purposes of illustration. The figures are provided for the purpose of illustrating one or more implementations with the explicit understanding that the figures will not be used to limit the scope or the meaning of the claims. It will also be recognized that the fluids disclosed herein are base oils and as such, may lack any wear protective additives until fully formulated with appropriate wear protection additives.

### DETAILED DESCRIPTION

Following below are more detailed descriptions of various concepts related to a dual-use coolant and lubricant and methods of manufacturing thereof. The methods, apparatuses, and systems introduced herein may be implemented in various ways, as the described concepts are not limited to any particular manner of implementation. Examples of specific implementations and applications are provided primarily for illustrative purposes.

Traditionally, electrified systems such as powertrains, data centers, fuel cell stacks, etc. are typically cooled by refrigerated air or liquid cooling wherein traditional ethylene-glycol water (EG-water) based coolants are circulated through cooling channels and do not contact the battery cells, battery tabs, or power inverters directly. Air cooling has been shown to be ineffective in applications with expected heat loads larger than a passenger-car type electric vehicle and are insufficient for heavy duty applications. Air cooling may not be suitable for thermal management systems due to low performance, excessive parasitic energy losses from fans, compressors, and additional weight. EG-water cooled systems have a risk of a fluid leak (potentially leading to fires and explosions) into the battery pack due to crash, seal failure, or corrosion. These types of systems further require high flow rates due to strict thermal demands and would require additional electrical insulation or heat transfer media to increase breakdown voltage in coolant channels to prevent electrical shorts.

Intrusion of water based coolants into the battery compartment can lead a thermal runaway by heat generated by electrical arcing, hydrogen generation by electrolysis of water, coolant reaction with the electrolyte, or from insufficient battery cell cooling. A thermal runaway event can originate in a single cell and where the internal temperature can increase several hundred degrees Celsius in a matter of seconds. This can cause a cascading failure to other adjacent battery cells and this event can rapidly lead to battery fires. With a coolant that cannot readily react with the electrolyte, is resistant to electrolysis, and is highly insulative, a thermal runaway can potentially be slowed down if not averted by preventing the event from spreading to other battery cells.

The above issues translate into lower effective battery power density and increased risk of battery failure or thermal runaway. Refrigerated cooling may also be used for electrified power systems but refrigerants are about 1.2-1.5× as dense as a traditional EG-water coolant. There would be a significant weight to performance tradeoff if direct-submersion refrigerant cooling was used to cool electrified powertrains due to additional cooling machinery and plumbing as well as parasitic energy to operate the compressor pump. Parasitic energy losses plus the additional weight from plumbing and equipment translates into a lower effective battery power density.

Furthermore, some electrified powertrain components, such as the traction drive motor, require a low-conductive lubricating fluid to provide cooling, lubrication, and wear protection of electrical windings and gear teeth. A commercially available automatic transmission fluid may be used for these functions. Oxidatively-stable organic fluids that possess low electrical conductivity, moderate viscosity, good heat capacity, freeze protection, lubrication, and anti-wear properties are desired to cool and lubricate electrified power train components. In the present disclosure, an organic fluid that possesses these properties can function as a dual-use coolant, or a base fluid for a lubricant. However, commercial transmission fluids suffer from lower heat transfer properties (i.e., heat capacity, thermal conductivity, etc.) than an EG-water coolant.

Various embodiments of the dual-use coolant may provide one or more benefits including, for example: (1) a good heat capacity; (2) an excellent breakdown voltage; (3) sufficient viscosity to lubricate and provide optimal heat transfer; (4) inexpensive feedstock materials; (5) oxidation resistance so as to maintain properties such as breakdown voltage, viscosity, and corrosion protection; (6) the ability to save weight in a vehicle; (7) a reduction in manufacturing costs; (8) low electrical conductivity; (9) freeze protection; (10) lubricating capability; (11) anti-wear properties; (12) use in electrified systems such as traction drive motors, thermal management systems, and fuel cell cooling systems; (13) the ability to greatly simplify fluid selection and service for electrified powertrains; (14) similar performance to conventional fluids used as lubricants and coolants; and (15) very low water solubility (e.g., less than 30 ppm water) so as to prevent moisture intrusion and contamination.

### I. Overview

A dual-use coolant comprises a fluid with a chemical formula corresponding to Structure I shown below.

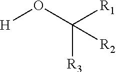

The fluid of Structure I is a tertiary alcohol. The fluid may comprise a R₁ group, a R₂ group, and a R₃ group. Using a fluid that can function both as a lubricant and a coolant may reduce weight in a vehicle and manufacturing costs. Further, the fluid that is used as both a coolant and a lubricant must have suitable properties for both functions without degradation of these properties over time.

### II. Example Dual-Use Coolant and Lubricant Fluid

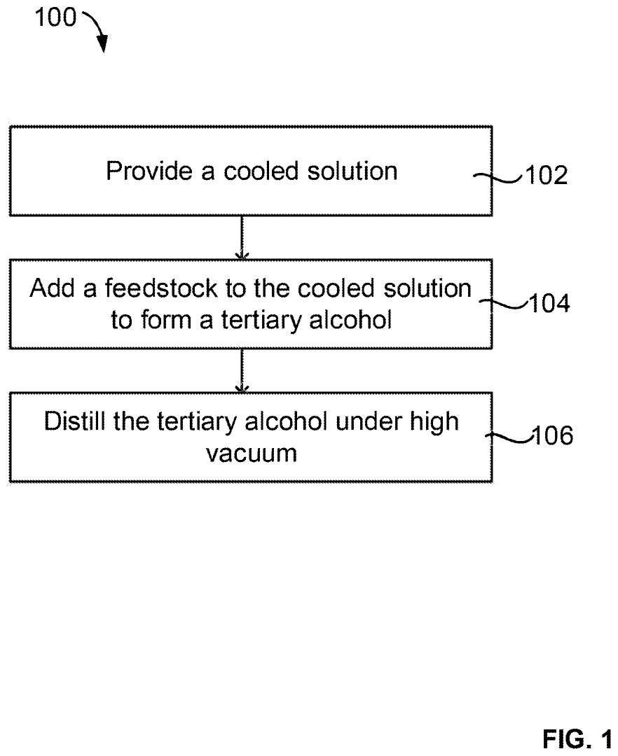

FIG. 1 illustrates a schematic flow diagram for an example method 100 of forming a lubricant, in accordance with some embodiments. The method 100 may start with operation 102 in which a cooled solution is provided. In some embodiments, the cooled solution may comprise methylmagnesium chloride, methylmagnesium bromide, methyl lithium, butyllithium cyclohexylmagnesium bromide, diethylzinc, cetyl zinc bromide, or any other suitable material of sufficient nucleophilicity to add to a ketone or ester. In some embodiments, the solution may be cooled by utilizing an external temperature control (e.g., an ice bath, a chiller, etc.) so as to prevent a runaway thermal event due to an exothermic reaction. In some embodiments, the cooled solution is a temperature in a range of about 10° C. to about 30° C. (e.g., 10° C., 15° C., 20° C., 25° C., or 30° C., inclusive). In some embodiments, the cooled solution is commercialized and is at a temperature outside a range of about 10° C. to about 30° C. In some embodiments, the cooled solution may include methlymagnesium chloride and may be added to a feedstock comprising an ester or a ketone. In such embodiments, the formed tertiary alcohol may be a fluid referred to as X-500, and X-500 may be suitable for use as a dual-use coolant.

The method 100 then continues to operation 104 in which a feedstock is added to the cooled solution to form a tertiary alcohol. The feedstock may be any suitable biomass, such as, but not limited to, triglyceride oil, biofuel, other commercially available esters, commercially available ketones, any combination thereof, or any other suitable material. In some embodiments, the feedstock may comprise cottonseed oil, vegetable oil, canola oil, coconut oil, biodiesel, flax seed oil, walnut oil, soybean oil, palm oil, peanut oil, olive oil, or any other suitable oil or triglyceride or any suitable combination thereof. In other embodiments, the feedstock may be a ketone instead of an ester. Combining the feedstock and the cooled solution may form a crude mixture containing the tertiary alcohol. In some embodiments, the formed tertiary alcohol may be extracted and concentrated.

The method 100 then continues to operation 106 in which the tertiary alcohol is distilled under high vacuum. Distillation may comprise separating components of a liquid mixture by using selective boiling and condensation. The tertiary alcohol may be distilled by any suitable distillation method such as dry distillation, simple distillation, fractional distillation, steam distillation, vacuum distillation, short path and molecular distillation, air-sensitive vacuum distillation, zone distillation, batch or differential distillation, continuous distillation, azeotropic distillation, industrial distillation, combinations thereof, or any other suitable distillation method. The high vacuum may be applied to the system containing the tertiary alcohol by any suitable method such as, but not limited to, providing a vacuum manifold and a vacuum pump. The high vacuum may be applied by any suitable device configured to provide a vacuum, such as a pump (e.g., a rotary vane pump, a diaphragm pump, a piston pump, a scroll pump, a screw pump, a Wankel pump, an external vane pump, any positive displacement pump, or any other suitable pump). In some embodiments, the tertiary alcohol after distillation may have a purity of greater than or equal to about 90% (e.g., 90%, 91%, 92%, 93%, 94%, 95%, 96%, 97%, 98%, or 99%, inclusive).

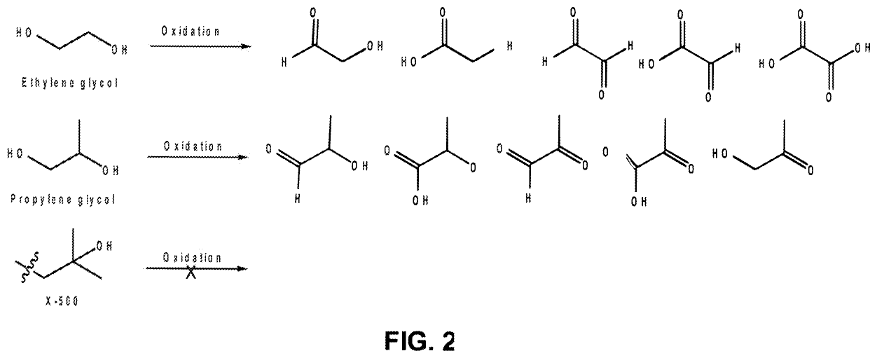

FIG. 2 demonstrates the chemical reaction of primary and secondary alcohols after oxidation. For example, ethylene glycol, an organic alcohol that has been previously known to be used as a coolant, oxidizes into carboxylic acids, formic acids, oxalic acids, and glycolic acids after exposure to oxygen in the powertrain system. As another example, propylene glycol, which also may be used as a coolant, also oxidizes into structures including carboxylic acids, formic acids, oxalic acids, and glycolic acids. Carboxylic acids, formic acids, oxalic acids, and glycolic acids are acidic in comparison to ethylene glycol and propylene glycol, which lead to corrosion and other problems that hinder coolant function. Further, primary alcohols (such as ethylene glycol) and secondary alcohols (such as propylene glycol) may have very poor breakdown voltage, particularly in comparison to tertiary alcohols. Breakdown voltage is the minimum voltage that causes a portion of a material to become electrically conductive across an electrode gap under conditions defined in ASTM D1816-12 and is a measure of a fluid's electrical insulating abilities. Primary and secondary alcohols may improve their breakdown voltage by including additives and/or antioxidants meant to combat oxidation, however, these additives can have a negligible or negative effect on insulating properties.

In contrast, tertiary alcohols, such as X-500 and its related compounds cannot be oxidized like primary and secondary alcohols. Therefore, the tertiary alcohol is more likely to remain as the parent molecule over time which maintains the high breakdown voltage. Further, tertiary alcohols may not require the addition of an additive and/or antioxidants, lessening the potential impact of these materials on breakdown voltage and heat capacity as well as reducing the cost of the fluid. Preventing the formation of oxidation products may also have positive effects on cooling system items such as piping and seals.

The fluid in the dual-use coolant may comprise the chemical formula of Structure I as shown below.

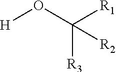

In some embodiments, R₁ in Structure I comprises, consists essentially of, or is selected from a group consisting of a branched or unbranched C₍₃₋₂₀₎ alkyl or allyl group. In such embodiments, R₁ may comprise, consist essentially of, or is selected from a group consisting of at least one hydrogen atom within the branched or unbranched C₍₃₋₂₀₎ alkyl or allyl group.

Expanding further, R₁ may comprise, consist essentially of, or is selected from a group consisting of a linear C₍₃₋₂₀₎ alkyl group, a branched C₍₃₋₂₀₎ chain, an unbranched alkene, a cyclic alkyl, or a heteroalkyl group. In some embodiments, the linear C₍₃₋₂₀₎ alkyl group in R₁ may comprise, consist essentially of, or is selected from a group consisting of at least one of unsubstituted alkanes, saturated alkanes, unsaturated alkanes, substituted alkanes, cyclic alkanes, or heterocyclic alkanes as well as aryl or heteroaryl groups. In such embodiments, the substituted alkanes may be substituted up to five times with any combination of at least two hydrogen atoms or at least one of OH, O—R₄, CN, NR₅R₆, or SO₍ₓ₎—R₇, wherein x is 0, 1, or 2. In some embodiments, the branched C₍₃₋₂₀₎ chain in R₁ may comprise, consist essentially of, or is selected from a group consisting of unsubstituted alkanes, or substituted alkanes substituted up to five times with any combination of at least two hydrogen atoms or at least one of OH, O—R₄, CN, NR₅R₆, or SO₍ₓ₎—R₇, wherein x is 0, 1, or 2. In some embodiments, the unbranched alkene in R₁ may be unsubstituted, or substituted up to three times with any combination of at least two hydrogen atoms or at least one of OH, O—R₄, CN, NR₅R₆, or SO₍ₓ₎—R₇, wherein x is 0, 1, or 2. In some embodiments, the cyclic alkyl in R₁ may be unsubstituted or substituted up to five times with any combination of a halogen, an aryl group, a heteroaryl group, or at least one of OH, O—R₄, CN, NR₅R₆, or SO₍ₓ₎—R₇, wherein x is 0, 1, or 2. In some embodiments, the heteroalkyl in R₁ may be unsubstituted or substituted with any combination of up to five times with any combination of a halogen, an aryl group, a heteroaryl group, or at least one of OH, O—R₄, CN, NR₅R₆, or SO₍ₓ₎—R₇, wherein x is 0, 1, or 2. R₄ may be present as any combination of R₁ as described above. R₅, R₆, and R₇ may or may not be present independently or simultaneously as H or C, or in any combination of R₁ as described above.

In some embodiments, the C₍₃₋₂₀₎ alkyl group of R₁ in Structure I comprises, consists essentially of, or is selected from a group consisting of a linear C₍₃₋₂₀₎ alkyl group. In such embodiments, the linear C₍₃₋₂₀₎ alkyl group may comprise, consist essentially of, or is selected from a group consisting of at least one of unsubstituted alkanes, saturated alkanes, unsaturated alkanes, substituted alkanes, cyclic alkanes, or heterocyclic alkanes as well as aryl or heteroaryl groups. In such embodiments, the substituted alkanes may be substituted up to five times with any combination of at least two hydrogen atoms or at least one of OH, O—R₄, CN, NR₅R₆, or SO₍ₓ₎—R₇, wherein x is 0, 1, or 2.

In some embodiments, the C₍₃₋₂₀₎ alkyl group of R₁ in Structure I comprises, consists essentially of, or is selected from a group consisting of a branched C₍₃₋₂₀₎ chain comprising at least one of unsubstituted alkanes, or substituted alkanes substituted up to five times with any combination of at least two hydrogen atoms or at least one of OH, O—R₄, CN, NR₅R₆, or SO₍ₓ₎—R₇, wherein x is 0, 1, or 2.

In some embodiments, the C₍₃₋₂₀₎ alkyl group of R₁ in Structure I comprises, consists essentially of, or is selected from a group consisting of at least one of an unbranched unsubstituted alkene, or an unbranched substituted alkene substituted up to three times with any combination of 0 to 2 hydrogen atoms or at least one of OH, O—R₄, CN, NR₅R₆, or SO₍ₓ₎—R₇, wherein x is 0, 1, or 2.

In some embodiments, the C₍₃₋₂₀₎ alkyl group of R₁ in Structure I comprises, consists essentially of, or is selected from a group consisting of at least one of an unsubstituted cyclic alkyl, a substituted cyclic alkyl, an unsubstituted heteroalkyl, or a substituted heteroalkyl. In such embodiments, the substituted cyclic alkyl may be substituted up to five times with any combination of a halogen, an aryl group, a heteroaryl group, or at least one of OH, O—R₄, CN, NR₅R₆, or SO₍ₓ₎—R₇, wherein x is 0, 1, or 2. In some embodiments, the substituted heteroalkyl is substituted up to five times with any combination of a halogen, an aryl group, a heteroaryl group, or at least one of OH, O—R₄, CN, NR₅R₆, or SO₍ₓ₎—R₇, wherein x is 0, 1, or 2.

In the fluid in the dual-use coolant, R₂ in Structure I may be a C₍₁₋₂₀₎ alkyl group and in any combination of R₁ as previously described herein. Further, R₃ in Structure I may also be a C₍₁₋₂₀₎ alkyl group and comprise any combination of R₁. In some embodiments, the R₁, R₂, and R₃ groups in Structure I may be the same or different from each other. It is understood that the fluid in the dual-use coolant is not limited to the structures listed above, and may comprise any suitable material in order to maintain suitable properties as a coolant after formulation with suitable anti-wear additives, and optionally as a base fluid for a lubricant as well.

The fluid in the dual-use coolant may comprise at least one of 3-Ethyl-pentan-3-ol, 3-Methyl-hexan-3-ol, 2-Methyl-pentan-2-ol, 2-Methyl-pentan-3-ol, 2-Cyclopropyl-pentan-2-ol, 1,1-Dicyclopropyl-pentan-1-ol, 2,4-Dimethyl-pentan-2-ol, 2-Cyclopentyl-4-methyl-pentan-2-ol, 2-Cyclohexyl-4-methyl-pentan-2-ol, 1-Cyclohexyl-1-cyclopropyl-3-methyl-butan-1-ol, 1-Cyclohexyl-1-cyclopropyl-3-methyl-pentan-1-ol, 1-Cyclohexyl-1-cyclopentyl-3-methyl-butan-1-ol, 1-Cyclohexyl-1-cyclopentyl-3-methyl-pentan-1-ol, 3-Ethyl-hexan-3-ol, 2-Methyl-hexan-2-ol, 2-Methyl-hexan-3-ol, 2,3-Dimethyl-hexan-3-ol, 3-Ethyl-2-methyl-hexan-3-ol, 3-Isopropyl-2-methyl-hexan-3-ol, 2,3-Dimethyl-hexan-3-ol, 2-Cyclopropyl-hexan-2-ol, 3-Cyclopropyl-hexan-3-ol, 1,1-Dicyclopropyl-hexan-1-ol, 2,4-Dimethyl-hexan-2-ol, 3,5-Dimethyl-hexan-3-ol, 3-Ethyl-5-methyl-hexan-3-ol, 3,5-Diethyl-hexan-3-ol, 1,1-Dicyclopropyl-4-methyl-hexan-1-ol, 3-Ethyl-2,5-dimethyl-hexan-3-ol, 3-Ethyl-2-methyl-hexan-3-ol, 3-Cyclopropyl-hexan-3-ol, 3-Cyclopentyl-hexan-3-ol, 3-Cyclopentyl-5-methyl-hexan-3-ol, 2-Cyclopentyl-4-methyl-hexan-2-ol, 2-Cyclohexyl-4-methyl-hexan-2-ol, 3-Cyclohexyl-5-methyl-hexan-3-ol, 3-Cyclohexyl-2,5-dimethyl-hexan-3-ol, 1-Cyclohexyl-1-cyclopropyl-3-methyl-hexan-1-ol, 1-Cyclohexyl-1-cyclopentyl-3-methyl-hexan-1-ol, or 4-Ethyl-3,5-diisopropyl-2,6-dimethyl-heptane-3,5-diol.

The fluid in the dual-use coolant may comprise at least one of 1,1-Dicyclohexyl-2-ethyl-hexan-1-ol, 3-Ethyl-heptan-3-ol, 2-Methyl-heptan-2-ol, 2-Methyl-heptan-3-ol, 2,4-Dimethyl-heptan-2-ol, 3-Methyl-heptan-3-ol, 3-Ethyl-heptan-3-ol, 2,3-Dimethyl-heptan-3-ol, 3-Ethyl-2-methyl-heptan-3-ol, 3-Isopropyl-2-methyl-heptan-3-ol, 2,3-Dimethyl-heptan-3-ol, 2-Cyclopropyl-heptan-2-ol, 3-Cyclopropyl-heptan-3-ol, 1,1-Dicyclopropyl-heptan-1-ol, 2,4-Dimethyl-heptan-2-ol, 3,5-Dimethyl-heptan-3-ol, 3-Ethyl-5-methyl-heptan-3-ol, 3,5-Diethyl-heptan-3-ol, 3,5-Dimethyl-heptan-3-ol, 1,1-Dicyclopropyl-4-methyl-heptan-1-ol, 3-Cyclopentyl-5-methyl-heptan-3-ol, 2-Cyclopentyl-4-methyl-heptan-2-ol, 2-Cyclohexyl-4-methyl-heptan-2-ol, 3-Cyclohexyl-5-methyl-heptan-3-ol, 3-Cyclohexyl-2,5-dimethyl-heptan-3-ol, 1-Cyclohexyl-1-cyclopentyl-3-methyl-heptan-1-ol, 2,4-Dimethyl-octan-2-ol, 3-Methyl-octan-3-ol, 3-Ethyl-octan-3-ol, 2-Methyl-octan-2-ol, 2-Methyl-octan-3-ol, 2,3-Dimethyl-octan-3-ol, 3-Ethyl-2-methyl-octan-3-ol, 3-Isopropyl-2-methyl-octan-3-ol, 2,3-Dimethyl-octan-3-ol, 2,3,6-Trimethyl-heptan-3-ol, 3,6-Dimethyl-heptan-3-ol, 3-Ethyl-2,5-dimethyl-heptan-3-ol, 2-Cyclohexyl-5-methyl-hexan-2-ol, 3-Methyl-4-propyl-heptan-4-ol, 4-Isopropyl-heptan-4-ol, 3-Isopropyl-2,4,6,6-tetramethyl-heptan-3-ol, 4-sec-Butyl-3,5,7,7-tetramethyl-octan-4-ol, or 4-Isopropyl-3,5,7,7-tetramethyl-octan-4-ol.

The fluid in the dual-use coolant may comprise at least one of 2-Cyclopropyl-octan-2-ol, 3-Cyclopropyl-octan-3-ol, 1,1-Dicyclopropyl-octan-1-ol, 2,4-Dimethyl-octan-2-ol, 3,5-Dimethyl-octan-3-ol, 3-Ethyl-5-methyl-octan-3-ol, 3,5-Diethyl-octnan-3-ol, 3,5-Dimethyl-octan-3-ol, 4,7-Dimethyl-octan-4-ol, 3-Cyclopropyl-6-methyl-octan-3-ol, 1,1-Dicyclopropyl-4-methyl-octan-1-ol, 3-Cyclopropyl-2,6-dimethyl-octan-3-ol, 2,3,6-Trimethyl-octan-3-ol, 3,5-Dimethyl-octan-3-ol, 3,4-Diethyl-octan-3-ol, 3-Cyclopropyl-5-methyl-octan-3-ol, 3-Ethyl-2,5-dimethyl-octan-3-ol, 3-Cyclopentyl-5-methyl-octan-3-ol, 2-Cyclopentyl-4-methyl-octan-2-ol, 3-Cyclohexyl-5-methyl-octan-3-ol, 3-Cyclohexyl-2,5-dimethyl-octan-3-ol, 1-Cyclohexyl-1-cyclopropyl-3-methyl-octan-1-ol, 1-Cyclohexyl-1-cyclopentyl-3-methyl-octan-1-ol, 3,4-Diethyl-2-methyl-octan-3-ol, 3-Ethyl-2,5-dimethyl-nonan-3-ol, 3-Cyclopentyl-5-methyl-nonan-3-ol, 2-Cyclopentyl-4-methyl-nonan-2-ol, 2-Cyclohexyl-4-methyl-nonan-2-ol, 3-Cyclohexyl-5-methyl-nonan-3-ol, 3-Cyclohexyl-2,5-dimethyl-nonan-3-ol, 1-Cyclohexyl-1-cyclopropyl-3-methyl-nonan-1-ol, 1,1-Dicyclopropyl-4-methyl-decan-1-ol, 3-Cyclopropyl-2,6-dimethyl-decan-3-ol, 4-Ethyl-3,6-dimethyl-octan-4-ol, 3,4,7-Trimethyl-octan-4-ol, 5-Ethyl-3,6-dimethyl-nonan-5-ol, 2,3-Dimethyl-nonan-3-ol, 5-(1-Hydroxy-1-isopropyl-2-methyl-propyl)-3,7-diisopropyl-2,8-dimethyl-nonane-3,7-diol, 3,7-Diethyl-5-(1-ethyl-1-hydroxy-propyl)-nonane-3,7-diol, 4,6-Di-sec-butyl-5-ethyl-3,7-dimethyl-nonane-4,6-diol, 4,5,6-Triethyl-3,7-dimethyl-nonane-4,6-diol, 3,7-Diethyl-5-(1-ethyl-1-hydroxy-2-methyl-propyl)-2,8-dimethyl-nonane-3,7-diol, 3,4-Dimethyl-decan-4-ol, 3,8-Diisopropyl-2,9-dimethyl-decane-3,8-diol, 3,8-Diethyl-decane-3,8-diol, or 3,8-Diethyl-2,9-dimethyl-decane-3,8-diol.

The fluid in the dual-use coolant may comprise at least one of 3-Ethyl-tetradecan-3-ol, 2-Methyl-tetradecan-2-ol, 2-Methyl-tetradecan-3-ol, 3-Cyclopropyl-6-methyl-hexadecan-3-ol, 1,1-Dicyclopropyl-4-methyl-hexadecan-1-ol, 3-Cyclopropyl-2,6-dimethyl-hexadecan-3-ol, 2,3,6-Trimethyl-hexadecan-3-ol, 4,7-Dimethyl-hexadecan-4-ol, 4-Ethyl-7-methyl-hexadecan-4-ol, 4-Ethyl-7-methyl-hexadecan-4-ol, 3,5-Dimethyl-hexadecan-3-ol, 3-Cyclopropyl-5-methyl-hexadecandecan-3-ol, 3-Ethyl-2,5-dimethyl-hexadecan-3-ol, 3-Cyclopentyl-5-methyl-hexadecan-3-ol, 2-Cyclopentyl-4-methyl-hexadeca-2-ol, 2-Cyclohexyl-4-methyl-hexadecan-2-ol, 3-Cyclohexyl-5-methyl-hexadecan-3-ol, 3-Ethyl-octadecan-3-ol, 2-Methyl-octadecan-2-ol, 3-Methyl-octadecan-3-ol, 3-Ethyl-octadecan-3-ol, 2,3-Dimethyl-octadecan-3-ol, 3-Ethyl-2-methyl-octadecan-3-ol, 3-Isopropyl-2-methyl-octadecan-3-ol, 2,3-Dimethyl-octadecan-3-ol, 2-Cyclopropyl-octadecan-2-ol, 3-Cyclopropyl-octadecan-3-ol, 1,1-Dicyclopropyl-octadecan-1-ol, 2,4-Dimethyl-octadecan-2-ol, 3,5-Dimethyl-octadecan-3-ol, 3-Ethyl-5-methyl-octadecan-3-ol, 3,5-Diethyl-octadecan-3-ol, 3,5-Dimethyl-octadecan-3-ol, 4-Ethyl-7-methyl-octadecan-4-ol, 3-Cyclopropyl-6-methyl-octadecan-3-ol, 1,1-Dicyclopropyl-4-methyl-octadecan-1-ol, 3-Cyclopropyl-2,6-dimethyl-octadecan-3-ol, 2,3,6-Trimethyl-octacan-3-ol, 4,7-Dimethyl-octadecan-4-ol, 4-Ethyl-7-methyl-octadecan-4-ol, 3,5-Dimethyl-octadecan-3-ol, 3-Cyclopropyl-5-methyl-octadecan-3-ol, 3-Ethyl-2,5-dimethyl-octadecan-3-ol, 3-Cyclopentyl-5-methyl-octadecan-3-ol, 2-Cyclopentyl-4-methyl-octadeca-2-ol, 2-Cyclohexyl-4-methyl-octadecan-2-ol, 3-Cyclohexyl-5-methyl-octadecan-3-ol, 3-Methyl-icosan-3-ol, 2-Methyl-icosan-2-ol, 3-Methyl-icosan-3-ol, 3-Ethyl-icosan-3-ol, 2,3-Dimethyl-icosan-3-ol, 3-Ethyl-2-methyl-icosan-3-ol, 3-Isopropyl-2-methyl-icosan-3-ol, 2-Cyclohexyl-4-methyl-icosan-2-ol.

The fluid in the dual-use coolant may comprise at least one of 4,8-Di-sec-butyl-6-(1-sec-butyl-1-hydroxy-2-methyl-butyl)-3,9-dimethyl-undecane-4,8-diol, 4,9-Di-sec-butyl-3,10-dimethyl-dodecane-4,9-diol, 4-Hexyl-3-isopropyl-2-methyl-dodecan-3-ol, 4,9-Dipropyl-dodecane-4,9-diol, 3-Isopropyl-2-methyl-dodecan-3-ol, 4-sec-Butyl-5-hexyl-3-methyl-tridecan-4-ol, 5-Hexyl-4-isopropyl-3-methyl-tridecan-4-ol, 3,12-Diisopropyl-2,13-dimethyl-tetradecane-3,12-diol, 3,14-Diisopropyl-2,15-dimethyl-hexadecane-3,14-diol, 4,13-Di-sec-butyl-3,14-dimethyl-hexadecane-4,13-diol, 4,13-Diisopropyl-3,14-dimethyl-hexadecane-4,13-diol, 5-sec-Butyl-2,2,4-trimethyl-heptadecan-5-ol, 3-Isopropyl-2-methyl-hexadecan-3-ol, 3,4-Diisopropyl-2-methyl-heptadecan-4-ol, 5-Isopropyl-2,2,4-trimethyl-heptadecan-5-ol, 4-sec-Butyl-3-methyl-heptadecan-4-ol, 4-Isopropyl-3-methyl-heptadecan-4-ol, 5-Ethyl-2,2,4-trimethyl-henicosan-5-ol, or 5-sec-Butyl-2,2,4-trimethyl-henicosan-5-ol.

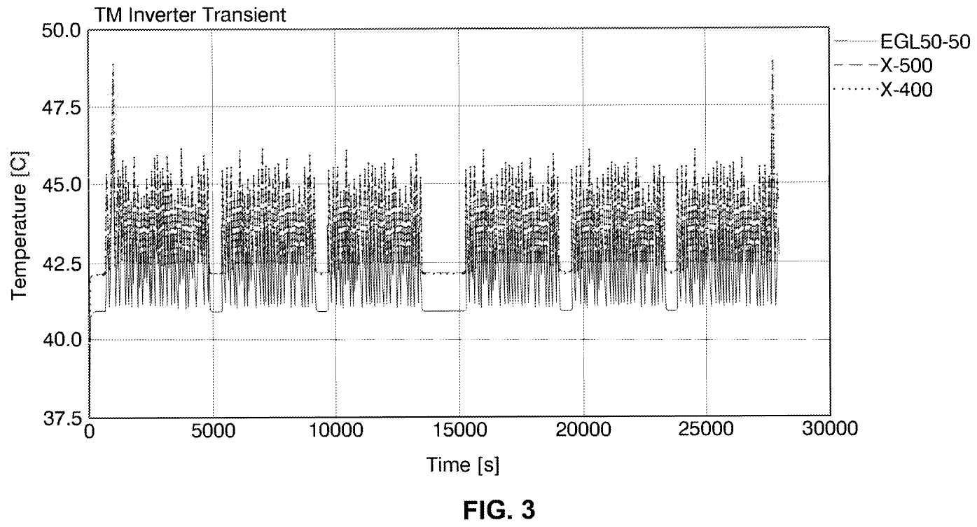

FIG. 3 illustrates a simulation of cooling the inverter temperature on a transit bus from an ethylene-glycol coolant and two dual-use coolants, X-500 and X-400. X-500 and X-400 are both tertiary alcohols containing the chemical structure of Structure I. The simulation in FIG. 1 occurs on a transit bus operating for several shifts including intermittent stops. An internal GT Power ETree model is used to model this simulation at a temperature of 38° C. and ambient pressure. As shown in FIG. 3, the X-500 is able to maintain the inverter temperature at 2° C. higher than the EG-water system (EGL50-50) with the same flow rate. This 2° C. increase is well below the derate threshold of 60° C., or the system limit beyond which power output, engine speed, or other variables could be reduced to protect vehicle systems, which illustrates that X-500 would be acceptable as a coolant. Further, the X-400 was able to maintain the inverter temperature at a temperature in between the EGL50-50 and the X-500, also demonstrating acceptability as a coolant. All of these simulations were performed in a model optimized for glycol coolants and not optimized for non-aqueous coolants. Therefore, it can be expected that the X-500 and X-400 will result in lower temperatures when the system is optimized for non-aqueous coolants.

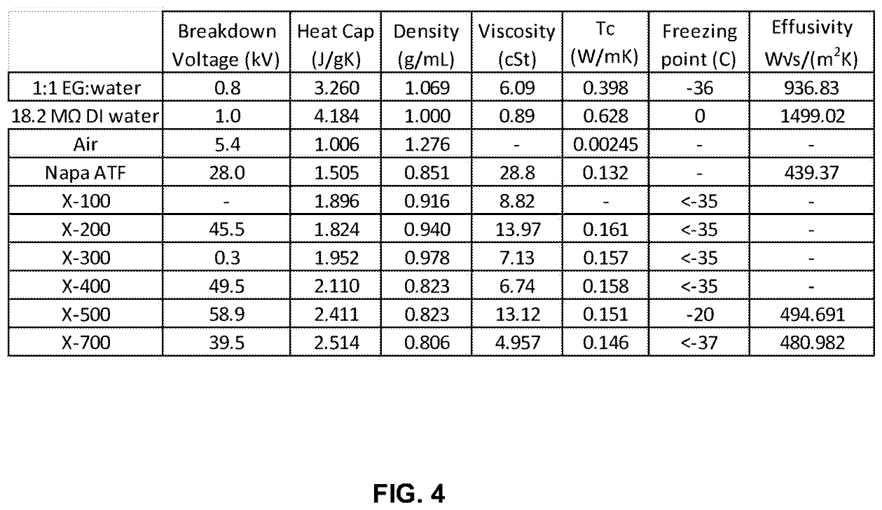

FIG. 4 illustrates a table of the properties of various coolants. X-100, X-200, X-300, X-400, X-500, X-600, and X-700. X-100, X-200, X-300, X-400, X-500, X-600, and X-700 are the coolants comprising the chemical structure of Structure I. The properties of Napa ATF (a commercial transmission fluid) are shown in FIG. 4. In the table, breakdown voltage is measured at a temperature in a range of 21° C. to 26° C. and according to ASTM D1816. Specific heat capacity is measured at 25° C. and according to ASTM E1269 or NIST values±1%. Density is measured at 40° C. according to ASTM D4052. If no literature value was available for viscosity, it was measured at 40° C. according to Southwest Research Institute (SWRI) and ASTM D445. If no literature was available for thermal conductivity (T_{c}), it was measured at 40° C. according to ASTM D7896. Freezing points were measured using a laboratory chiller capable of attaining a temperature of −40° C. Effusivity was measured by a MTPS sensor with a C-Therm Modified Transient plane source and is an average of 10 measurements recorded at ambient temperature.

The X-500 tertiary alcohol meets all of the desired properties of a coolant, with the exception of the freezing point requirement. Although X-500 does not provide as much freeze protection as may be desired (e.g., −40° C.), it still provides adequate freeze protection for the majority of relevant scenarios. The X-500 in the table of FIG. 4 is about 92% pure, and an impurity in X-500 from manufacturing of the X-500 coolant increases the freezing point thereof. As such, if the purity were to increase, the freezing point would decrease, which is desired. As shown in FIG. 4, X-500 has a higher breakdown voltage in a range of about 50 to about 65 kV (e.g., 50 kV, 55 kV, 60 kV, or 65 kV, inclusive) at 1 mm gap which is much higher than the current industry standard of EG-water at 0.8 kV. ASTM D-3487 calls for insulating fluids to have a breakdown voltage of at least 20 kV to be acceptable.

X-500 has a heat capacity in a range of about 2 to about 3.1 J/g° C. (e.g., 2 J/g° C., 2.2 J/g° C., 2.4 J/g° C., 2.6 J/g° C., 2.8 J/g° C., or 3 J/g° C., inclusive) which is lower than the heat capacity of EG-water which has a heat capacity in a range of about 3.2 to about 3.7 J/g° C. (e.g., 3.2 J/g° C., 3.3 J/g° C., 3.4 J/g° C., 3.5 J/g° C., 3.6 J/g° C., or 3.7 J/g° C., inclusive). However, as shown in FIG. 3, X-500 is still able to cool the individual components to a satisfactory level. X-500 has a density in a range of about 0.75 to about 1 g/mL (e.g., 0.75 g/ml, 0.8 g/mL, 0.85 g/mL, 0.9 g/mL, 0.95 g/mL, or 1 g/mL, inclusive) which is less than the density of EG-water, possibly reducing the weight of coolant used.

X-500 also has a viscosity in a range of about 12 to about 15 cSt (e.g., 12 cSt, 13 cSt, 14 cSt, or 15 cSt, inclusive) which is greater than the viscosity of EG-water at about 6.09 cSt. Finally, X-500 has a freezing point in a range of −30° C. to about −10° C. (e.g., −30° C., −25° C., −20° C., −15° C., or −10° C., inclusive) which is higher than the freezing point of EG-water. However, it is possible to lower the freezing point of X-500 by increasing the purity of the fluid or with the addition of appropriate additives.

Therefore, the properties of X-500 as shown by FIG. 4 demonstrate that the fluid is capable of performance similar to conventional automatic transmission fluids that are currently used to lubricate and cool automatic transmission components. X-500 is also capable of resisting changes in breakdown voltage and insulating ability as a result of moisture intrusion and contamination. Despite X-500 having a lower heat capacity than EG-water, it has a far superior breakdown voltage and a higher viscosity. Further, X-500 is derived from an inexpensive, safe, and readily available feedstock. The fluid X-500, or any other suitable fluid made from the method 100 and containing the chemical Structure I may be used in a multitude of capacities such as, but not limited to, low-conductivity systems, dielectric systems, as a coolant, as a coolant additive, as a lubricant, in wear reduction, in transmission, in automatic transmission fluids, in batteries, in fuel cells, in battery thermal management systems, in traction motors, as freeze protection, in insulation, or in electrified powertrains.

### III. Example Methods of Making the Coolant

Methylmagnesium chloride in diethyl ether (3M, 4810.455 mmol) was charged into a dry nitrogen purged, jacketed 5 L reactor fitted with a thermometer, addition funnel, and mechanical stirring. The solution was cooled to between 0° C. and 10° C. To this stirred solution, the appropriate ester or ketone (534 mmol) was charged at such a rate as to maintain the solution temperature below 25° C. to prevent boiling of the solvent. After the addition was complete, the solution was allowed to return to ambient temperature and aged with stirring for one hour after which it was cooled to 10° C. To this was added deionized water (ca 1 L) at such a rate as to maintain the temperature under 25° C. until the solution was now a thick suspension at which time, concentrated hydrochloric acid was added (6948.436 mmol) at such a rate as to maintain the temperature below 20° C. Cooling was maintained until the addition of the acid was complete then additional water (1.5 L) was added. Cooling was removed and the biphasic suspension was allowed to stir until the solids had all dissolved and the pH of the aqueous was found to be <7. The water was removed and the remaining yellow ethereal solution was washed with additional dilute HCl (500 mL), saturated bicarbonate (2×1 L), and brine (2 L). The remaining ether was then dried with anhydrous sodium sulfate, filtered, and removed to afford a yellow oil.

The yellow oil was fractionally distilled under high vacuum in a Hemple column packed with 12 mm Raschig rings. The purity was found to be between 75-90% and an additional distillation afforded a clear oil found to be >92% purity by gas chromatography. Additional procedure that can precede the above if the organometallic reagent is not commercially available: Magnesium or lithium turnings were dried under argon with slow stirring for three days in a 3 liter, 3 neck flask outfitted with a thermometer, addition funnel, and mechanical stirring. An anhydrous ethereal solvent (1.3 L) was transferred via cannula over the turnings and cooled to 0° C. with stirring. To this cooled suspension was added an organohalide (4046 mmol) added dropwise as to maintain a temperature below 30° C. After addition, the cooling was removed and the suspension was stirred for three hours then transferred via cannula to the vessel listed in the first procedure and the procedure is followed.

It should be appreciated that while particular methods for formulating the coolant according to the present disclosure are described herein, it should be appreciated that these methods are just examples and other methods for formulating the coolant are described herein and should be considered to be within the scope of the present disclosure.

### III. Construction of Example Embodiments

While this specification contains many specific implementation details, these should not be construed as limitations on the scope of what may be claimed but rather as descriptions of features specific to particular implementations. Certain features described in this specification in the context of separate implementations can also be implemented in combination in a single implementation. Conversely, various features described in the context of a single implementation can also be implemented in multiple implementations separately or in any suitable subcombination. Moreover, although features may be described as acting in certain combinations and even initially claimed as such, one or more features from a claimed combination can, in some cases, be excised from the combination, and the claimed combination may be directed to a subcombination or variation of a subcombination.

As utilized herein, the terms “generally,” “substantially,” “similarly,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

The term “coupled” and the like, as used herein, mean the joining of two components directly or indirectly to one another. Such joining may be stationary (e.g., permanent) or moveable (e.g., removable or releasable). Such joining may be achieved with the two components or the two components and any additional intermediate components being integrally formed as a single unitary body with one another, with the two components, or with the two components and any additional intermediate components being attached to one another.

It is important to note that the construction and arrangement of the various systems shown in the various example implementations is illustrative only and not restrictive in character. All changes and modifications that come within the spirit and/or scope of the described implementations are desired to be protected. It should be understood that some features may not be necessary, and implementations lacking the various features may be contemplated as within the scope of the disclosure, the scope being defined by the claims that follow. When the language “a portion” is used, the item can include a portion and/or the entire item unless specifically stated to the contrary.

What is claimed is:

1. A dual-use coolant comprising:
a fluid, the fluid having the following chemical formula:

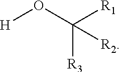

2. The coolant of claim 1, wherein R₁ is a C₍₃₋₂₀₎ alkyl group.

3. The coolant of claim 2, wherein R₁ comprises at least one hydrogen atom.

4. The coolant of claim 2, wherein R₁ comprises at least one of OH, O—R₄, CN, NR₅R₆, or SO₍ₓ₎—R₇, wherein x is 0, 1, or 2, where R₄ is any combination of R₁, and R₅, R₆, and R₇ are present independently or simultaneously as H or C, or in any combination of R₁.

5. The coolant of claim 2, wherein R₁ comprises a linear C₍₃₋₂₀₎ alkyl group comprising at least one of:
unsubstituted alkanes;
saturated alkanes;
unsaturated alkanes;
cyclic alkanes;
heterocyclic alkanes; or
substituted alkanes substituted up to five times with any combination of at least two hydrogen atoms or at least one of OH, O—R₄, CN, NR₅R₆, or SO₍ₓ₎—R₇, wherein x is 0, 1, or 2.

6. The coolant of claim 2, wherein R₁ comprises a branched C₍₃₋₂₀₎ chain comprising at least one of unsubstituted alkanes, or substituted alkanes substituted up to five times with any combination of at least two hydrogen atoms or at least one of OH, O—R₄, CN, NR₅R₆, or SO₍ₓ₎—R₇, wherein x is 0, 1, or 2.

7. The coolant of claim 2, wherein R₁ comprises at least one of an unbranched unsubstituted alkene, or an unbranched substituted alkene substituted up to three times with any combination of 0 to 2 hydrogen atoms or at least one of OH, O—R₄, CN, NR₅R₆, or SO₍ₓ₎—R₇, wherein x is 0, 1, or 2.

8. The coolant of claim 2, wherein R₁ comprises at least one of:
an unsubstituted cyclic alkyl;
a substituted cyclic alkyl substituted up to five times with any combination of:
a halogen,
an aryl group,
a heteroaryl group, or
at least one of OH, O—R₄, CN, NR₅R₆, or SO₍ₓ₎—R₇, wherein x is 0, 1, or 2;
an unsubstituted heteroalkyl; or
a substituted heteroalkyl substituted up to five times with any combination of:
a halogen,
an aryl group,
a heteroaryl group, or
at least one of OH, O—R₄, CN, NR₅R₆, or SO₍ₓ₎—R₇, wherein x is 0, 1, or 2.

9. The coolant of claim 1, wherein R₂ comprises at least one of:
(i) a linear C₍₁₋₂₀₎ alkyl group comprising at least one of:
unsubstituted alkanes;
saturated alkanes;
unsaturated alkanes;
cyclic alkanes,
heterocyclic alkanes, or
substituted alkanes substituted up to five times with any combination of at least two hydrogen atoms or at least one of OH, O—R₄, CN, NR₅R₆, or SO₍ₓ₎—R₇;
(ii) a branched C₍₁₋₂₀₎ alkyl chain comprising at least one of unsubstituted alkanes, or substituted alkanes substituted up to five times with any combination of at least two hydrogen atoms or at least one of OH, O—R₄, CN, NR₅R₆, or SO₍ₓ₎—R₇;
(iii) at least one of an unbranched unsubstituted alkene, or an unbranched substituted alkene substituted up to three times with any combination of 0 to 2 hydrogen atoms or at least one of OH, O—R₄, CN, NR₅R₆, or SO₍ₓ₎—R₇;
(iv) an unsubstituted cyclic alkyl;
(v) a substituted cyclic alkyl substituted up to five times with any combination of:
a halogen,
an aryl group,
a heteroaryl group, or
at least one of OH, O—R₄, CN, NR₅R₆, or SO₍ₓ₎—R₇;
(vi) an unsubstituted heteroalkyl; or
(vii) a substituted heteroaryl substituted up to five times with any combination of:
a halogen,
an aryl group,
a heteroaryl group, or
at least one of OH, O—R₄, CN, NR₅R₆, or SO₍ₓ₎—R₇,
wherein x is 0, 1, or 2.

10. The coolant of claim 1, wherein R₃ comprises at least one of:
(i) a linear C₍₁₋₂₀₎ alkyl group comprising at least one of:
unsubstituted alkanes;
saturated alkanes;
unsaturated alkanes;
cyclic alkanes;
heterocyclic alkanes; or
substituted alkanes substituted up to five times with any combination of at least two hydrogen atoms or at least one of OH, O—R₄, CN, NR₅R₆, or SO₍ₓ₎—R₇;
(ii) a branched C₍₁₋₂₀₎ chain comprising at least one of unsubstituted alkanes, or substituted alkanes substituted up to five times with any combination of at least two hydrogen atoms or at least one of OH, O—R₄, CN, NR₅R₆, or SO₍ₓ₎—R₇;
(iii) at least one of an unbranched unsubstituted alkene, or an unbranched substituted alkene substituted up to three times with any combination of 0 to 2 hydrogen atoms or at least one of OH, O—R₄, CN, NR₅R₆, or SO₍ₓ₎—R₇;
(iv) an unsubstituted cyclic alkyl;
(v) a substituted cyclic alkyl substituted up to five times with any combination of:
a halogen,
an aryl group,
a heteroaryl group, or
at least one of OH, O—R₄, CN, NR₅R₆, or SO₍ₓ₎—R₇;
(vi) an unsubstituted heteroalkyl; or
(vii) a substituted heteroalkyl substituted up to five times with any combination of:
a halogen,
an aryl group,
a heteroaryl group, or
at least one of OH, O—R₄, CN, NR₅R₆, or SO₍ₓ₎—R₇,
wherein x is 0, 1, or 2.

11. The coolant of claim 1, wherein R₂ and R₃ are connected by a C2-C4 bridge.

12. The coolant of claim 1, wherein the fluid has the following chemical formula:

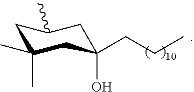

13. The coolant of claim 1, wherein the coolant is also used as base fluid for a lubricant.

14. A method, comprising:
providing a cooled solution;
adding a feedstock to the cooled solution to form a tertiary alcohol; and
distilling the tertiary alcohol under vacuum.

15. The method of claim 14, wherein the cooled solution comprises at least one of methylmagnesium chloride, methylmagnesium bromide, methyl lithium, butyllithium cyclohexylmagnesium bromide, diethylzinc, or cetyl zinc bromide.

16. The method of claim 14, wherein the feedstock comprises a biomass.

17. The method of claim 14, wherein the tertiary alcohol has the following chemical formula: 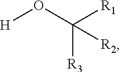
and
wherein R₁ is a C₍₃₋₂₀₎ alkyl group

18. The method of claim 14, wherein the tertiary alcohol has the following chemical formula: 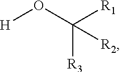
and
wherein R₂ and R₃ are connected by a C2-C4 bridge.

19. The method of claim 14, wherein the tertiary alcohol has the following chemical formula:

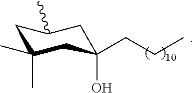

20. The method of claim 14, wherein the tertiary alcohol after distillation has a purity of greater than or equal to 90%.
